(19) United States
(12) Patent Application Publication
(10) Pub. No.: US 2023/0256448 A1
(43) Pub. Date: Aug. 17, 2023

(54) IMPROVEMENTS TO APPARATUS AND METHODS FOR MANIPULATING MICRODROPLETS

(71) Applicant: LIGHTCAST DISCOVERY LTD, Cambridge, Cambridgeshire (GB)

(72) Inventors: Maciej SOSNA, Cambridge (GB); Evangelia-Nefeli ATHANASOPOULOU, Cambridge (GB); Mei WU, Cambridge (GB)

(73) Assignee: LIGHTCAST DISCOVERY LTD, Cambridge, Cambridgeshire (GB)

(21) Appl. No.: 17/925,238

(22) PCT Filed: May 14, 2021
(86) PCT No.: PCT/GB2021/051168

§ 371 Date: Nov. 14, 2022

(30) Foreign Application Priority Data
May 15, 2020 (GB) 2007249.2

(51) Int. Cl. B01L 3/00

(52) U.S. Cl. CPC B01L 3/502792; B01L 3/502761; B01L 2200/0652

(57) ABSTRACT

A method of handling an adherent cell in a microdroplet assaying system by conjugating an adherent cell to a microbead is provided. The method 50 comprises the steps of: loading a first plurality of microdroplets into a microfluidic space, wherein each of the first microdroplet 5 contains a microbead 52 and a first fluid; loading a second plurality of microdroplets into the microfluidic space, wherein each of the second microdroplet contains an adherent cell and a second fluid 54; merging the first plurality of microdroplets and the second plurality of microdroplets to form a plurality of merged microdroplets 56, each merged microdroplets containing the first and second fluids, at least one microbead and at least one adherent cell; and10 agitating each of the merged microdroplets 58 to cause the first and second fluids in each of the merged microdroplets to move such that at least one adherent cell adhere to the at least one microbead. [FIG. 1] 15

The present invention relates to a method and a system for manipulating microdroplets and in particular to a method and system of handling cells in a microdroplet assaying system. The present invention also relates to a method of handling adherent cells in a microdroplet assaying system by conjugating adherent cells to microbeads.

Cells derived from human and/or animal tissues can be manipulated in culture for use as a research development tool, particularly for the production of viral vectors and vaccines, and various therapeutic proteins, in order to generate functional cells or tissue analogues for screening of medicines.

Mammalian cells can be made to produce medicines through viral infection, and therapeutic proteins through genetic engineering. Many of these medicines are necessary for patients who either lack the normal form of a protein or cannot produce it in sufficient quantity.

Such cell growth requires a complex environment containing a mixture of nutrients, including sugars, amino acids, vitamins, minerals, and growth factors such as insulin and various cytokines. Further, except for certain cell types in native to the bloodstream or lymphatic system, cells derived from tissues are anchorage-dependent, meaning they do not grow as free-floating individual cells. Therefore, after being released from the tissue environment, cells require a surface on which they can adhere, otherwise they will fail to survive and divide.

Methods of culturing adherent cells inside conventional microfluidics are known, for example, in organ-on-chip applications. In particular, there are existing plate-based workflows that attempt to culture adherent cells. Such microfluidic systems can include continuous-flow microfluidics in which streams of fluid are routed and controlled by micro-structured channels. It can also include digital microfluidics, in which discrete volumes of fluid are manipulated individually. One class of digital microfluidic comprises ‘ElectroWetting On Dielectric’ (EWOD) devices, and the sub-class of Optical EWOD devices such as those disclosed in WO2018/234445, which is incorporated by reference. Such devices allow controlled movement of droplets of cell media, optionally surrounded by an oil-based carrier phase, around a microfluidic chip. However, in order to achieve adherent cell growth on such platforms, it is necessary to controllably introduce contact between the droplet contents and some kind of culturing region on a chip device, which is complex to achieve in any conventional droplet handling microfluidics platform.

There has been limited disclosure of culturing adherent cells on digital microfluidics. For example, previous disclosures have described using conventional electrowetting in a lab-on-a-chip platform with an array of electrodes to implement mammalian cell culturing. However such platforms are limited in the number of cells they can manipulate simultaneously due to the large size of the fixed electrodes used for actuation. For the same reason such platforms are not generally suitable for manipulating individual cells encapsulated in sub-nanolitre volumes. Furthermore, with fixed electrode locations and sizes, the flexibility and adaptability of such systems is limited.

In other developments, adhesion of cells onto solid supports formed of microbead carriers is well known and is conventionally used for scaled-up bio-production where the microbeads provide an increased surface area for cell adhesion. However, combining solid microbeads with adherent cells using conventional droplet platforms is challenging, especially working at the single cell level. It is not possible to perform a large number of droplet merges in parallel with control such that each cell is exposed to a precise number of beads. When culturing adherent cell lines, it is important to be able to control the precise number of beads each cell is exposed to, because the growth of adherent cells is dependent on the available surface area on the adhered support. Therefore the number of cells adhered to each microbead must be precisely controlled for an optimal adherent cell culture. Instruments where droplets are incubated or stored in channels have poor levels of cell viability owing to restricted supply of gases.

For many important assays, after adherent cells are screened for phenotypic traits they must be recovered from the microfluidic system. In some assays this is for the purpose of conducting genetic analyses, which may include DNA sequencing, RNA sequencing or PCR detection. In some assays this recovery is for the purposes of expanding colonies of cells from the recovered material, including the case where a clonal colony is to be expanded from a single recovered cell. When the adherent cells are required to grow in to colonies they must enter the adherent state after recovery. In any other microfluidic system where adherent cells are grown on-chip and recovered for expansion, they must temporarily be taken back in to suspension state for the transportation out of the chip, and then returned to the adherent state for growth in microwell plates or flasks. Every time a cell is taken between the adherent and suspension states the process induces stresses to the cell, reduces the cell viability and changes the expression profile of the cells. As such the process of recovery and associated re-suspension is a known deficiency of microfluidic cell culture systems, and it is highly advantageous to have a system where cells can be recovered from a microfluidic system in their adherent state.

Therefore, there is a requirement to provide a method and apparatus for attaching, growing and/or dispensing adherent cells onto beads, such as microbeads, in an efficient, quick and cost-effective manner. The method and apparatus should maintain sufficient control during the droplet merge step such that each adherent cell is exposed to a precise number of beads, even when large numbers of droplets are merged in parallel. By providing an efficient method to control the attachment to microbeads and detachment from microbead, of adherent cells, it can promote cellular attachment and proliferation to enable controlled growth of target cells in a high throughput environment.

It is against the background that the present invention has arisen.

According to an aspect of the present invention, there is provided a method of handling an adherent cell in a microdroplet assaying system by conjugating an adherent cell to a microbead, the method comprising: loading a first plurality of microdroplets into a microfluidic space, wherein each of the first microdroplet contains a microbead and a first fluid; loading a second plurality of microdroplets into the microfluidic space, wherein each of the second microdroplet contains an adherent cell and a second fluid; merging the first plurality of microdroplets and the second plurality of microdroplets to form a plurality of merged microdroplets, each merged microdroplets containing the first and second fluids, at least one microbead and at least one adherent cell; and agitating each of the merged microdroplets to cause the first and second fluids in each of the merged microdroplets to move such that at least one adherent cell adheres to the at least one microbead.

In some embodiments, there is provided a method of handling adherent cells in a microdroplet assaying system by conjugating adherent cells to microbeads, the method comprising: loading a first plurality of microdroplets containing microbeads and a first fluid and a second plurality of microdroplets containing adherent cells and a second fluid into a microfluidic space; merging the first plurality of microdroplets and the second plurality of microdroplets to form a plurality of merged microdroplets, each merged microdroplet containing the first and second fluids, at least one microbead and at least one adherent cell; and agitating the merged microdroplets to cause the first and second fluids to move such that at least one adherent cell adheres to the at least one microbead.

The adherent cells may be temporarily retained in a suspended state before culturing.

Optionally, ejecting the merged microdroplets from the microfluidic space and dispensing them on to a treated microwell plate where the cells are caused to attach to the plate and proliferate.

The method as disclosed in the present invention is advantageous because it provides an efficient and scalable method to control and promote the attachment to microbeads and detachment from microbeads, of adherent cells. Thus, the method of the present invention allows the user to reliably control and/or manipulate the growth of target cells such as mammalian cells.

The term ‘adherent cells’ as used within the context of this invention, should be understood to include any cell line which requires a supporting structure for cell viability during culture. Other types of cells can grow freely in suspension, and do not require a solid support for growth and proliferation. Adherent cells are anchorage dependent and require adhesion to a solid support in order to grow. Adherent cells may be adapted for growth in suspension. However this requires transitioning the adherent cells to a suspension state, which reduces cell viability. The term ‘adherent cells’ should therefore be understood to be distinct from cell lines which do not require adherence to a solid support for cell viability, but which can be attached to a support for other reasons, such as for use as assay reporters. Examples of adherent cells include, but are not limited to, mammalian tissue cells such as Chinese Hamster Ovary (CHO) cells, production cell lines, epithelial cells and certain types of cancer cells.

In addition, adhering adherent cells to at least one microbead is highly desirable because microbeads can provide an increased surface area for adherent cell adhesion which is particularly useful for scaled-up bio production of adherent cells.

Moreover, microbeads provide a suitable substrate on which adherent cells could bind in order to survive, proliferate and express their conventional phenotypes. In addition, microbead carriers are advantageous to use as they can be easily manipulated and/or transported. In contrast, growing cells in a conventional droplet manipulation device require complex patterning of the devices to provide hydrophilic patches on the device where cells can adhere within a droplet which is fully wetted to the surface. This means that cells cannot be easily and readily transported or manipulated once they have entered an adherent state on the patches, as the cells are bound to the surface and the droplets are wetted to the patch.

Agitating the merged microdroplets is required to cause sufficient fluid flow for the adherent cell and the microbeads to come together within a period of a few minutes; cells and carrier beads are both slow-diffusing large particles and are unlikely to encounter each other through random diffusion and in stationary droplets there is minimal internal flow. Some systems rely on flowing microdroplets containing cells past microbeads in a single direction, however this can be insufficient to ensure the cell and the microbead combine. Agitating the merged microdroplets may come in the form of stirring the microdroplets or by shaking or by any other means capable to cause sufficient internal fluid flow for the adherent cell and the microbeads to come into contact. Agitation is not limited to movement in a single direction, and consequently the bead can move back-and-forth through the centre of the microdroplet multiple times during the agitation step, which ensures that the at least one adherent cell adheres to the at least one microbead. Both the cells and the droplets can enter the fastest flow stream inside the droplet and come into contact whilst they are in the narrow fast-flowing internal flow stream. For most agitation pattern this region of maximum flow is around the outside perimeter of the droplet.

The first and second fluids may be the same fluid contained in the first and second plurality of microdroplets, respectively. Alternatively, the first and the second fluids may be different. The first and/or second fluid(s) may be a fluid comprising a buffer suitable for promoting adhesion. Additionally or alternatively, the first and/or second fluid(s) may comprise cell growth media. Additionally or alternatively the first and/or second fluid(s) may comprise drugs, assay reagents, suspended viral vectors, biopolymers and gels.

The assaying system comprises a device which can be useful for manipulating microdroplets. The device may comprise a microfluidic chip adapted to receive and manipulate microdroplets dispersed in a carrier fluid flowing along pathways on a surface of the chip, wherein the microdroplets are manipulated using an optically-mediated electrowetting (oEWOD) force.

Manipulating microdroplets with oEWOD force is advantageous over other methods of microdroplet control such as trapping microdroplets with a physical structure, which can be inefficient and waste space on the chip. Some methods known in the art require the use of magnetic microbeads and a magnetic field in order to hold the microbead stationary for the merge step; however the present invention is suitable for use with a wide range of microbead materials. Microbeads with optimal characteristics for adherent cell culturing such as a high surface area to volume ratio and a high loading capability can therefore be selected. Alternatively, or in addition, the method is also suitable for use with microbeads of various shapes and aspect ratios, including rectangular or disc-shaped beads with a very high aspect ratio. In some embodiments the beads within the droplets are caused to move within the fluid through an external magnetic force applied to each microbead. Flat, high-aspect-ratio microbeads may be particularly suitable for manipulation in this way owing to their reduced susceptibility to aggregation effects.

Alternatively, or in addition, the method of the present invention is suitable for use with microbeads that are formed from a layer structure and have layers comprising magnetised and non-magnetised materials. Additionally, the method of the present invention is suitable for use with microbeads which display a barcode or marking pattern to aid the identification of specific beads. In some embodiments, the microbeads may comprised of a gel or a hydrogel. Gels can be advantageous as it can be used for supplying growth factors or nutrients to boost cell viability.

Furthermore, oEWOD force may be used to deliver reagents, cells and other materials to microbeads which are disposed on a surface. The microbeads may be independently retained or manipulated by an external magnetic force.

The method of the present invention may further comprise the step of performing a selection, assaying, culturing or recovery process on the at least one adherent cell adhered to the at least one microbead.

In some embodiments, the selection process may include, but is not limited to selecting only those cells expressing a fluorescent endogenous reporter, or select only those cells which exhibit signal in presence of a surface marker stain or select only those cells which assume a particular morphology or conformation around the bead.

In some embodiments, the selection process may include, but is not limited to, adding one or more reporter bead elements to the droplet via an additional merge operation, and monitoring the formation of a fluorescence signal around the reporter beads induced by coalescence of protein secreted by the adherent cells on to the bead in conjunction with a fluorescent reporter molecule and then subsequently selecting only those cells which secrete material picked up by a particular class of bead.

In some embodiments, performing an assaying process may include but is not limited to adding a drug and monitoring or identifying a response in the cell such as apoptosis or cell death, or adding a second population of cells or a single cell which acts upon the target cell bound to the bead, or adding a viral vector or merging in a stimulus such as a cytokine or other compound.

In some embodiments, performing a culturing process may include but is not limited to culturing cells to proliferate and grow across the bead or culturing cells in a range of different conditions such as different nutrients, cytokines and drugs added to each droplet during culture.

In some embodiments, performing a recovery process may be desirable to recover a microdroplet of interest for example, a microdroplet containing the microbead and/or adherent cells. The microdroplet recovered may be dispensed onto a plate such as a tissue culture treated well plate for further experiments.

The microfluidic chip may comprise a coating structure in which the microfluidic chip can be configured to manipulate microdroplets and to allow controlled attachment and detachment of adherent cells contained within the microdroplets by application of optically mediated electrowetting (oEWOD) force.

In some embodiments, the microfluidic space is part of a microfluidic chip configured to manipulate microdroplets via optically mediated electrowetting (oEWOD).

In some embodiments, the microfluidic space is part of a microfluidic chip configured to manipulate the first and second plurality of microdroplets via optically mediated electrowetting (oEWOD).

In some embodiments, the microfluidic chip of the present invention comprises oEWOD structures including first and second composite walls. The first composite wall may comprise a first substrate; the first substrate comprising a first transparent conductor layer on the substrate, the first transparent conductor layer having a thickness in the range 70 to 250 nm; a photoactive layer activated by electromagnetic radiation in the wavelength range 400-1000 nm on the conductor layer, the photoactive layer having a thickness in the range 300-1500 nm and a first dielectric layer on the photoactive layer, the first dielectric layer having a thickness in the range 30 to 160 nm. The second composite wall may comprise: a second substrate; a second conductor layer on the substrate, the second conductor layer having a thickness in the range 70 to 250 nm and optionally a second dielectric layer on the second conductor layer, the second dielectric layer having a thickness in the range 120 to 160 nm. The exposed surfaces of the first and second dielectric layers may be disposed less than 180 µm apart to define a microfluidic space adapted to contain microdroplets. An A/C source may be included to provide a voltage across the first and second composite walls connecting the first and second conductor layers. At least one source of electromagnetic radiation may also be provided having an energy higher than the bandgap of the photoactive layer adapted to impinge on the photoactive layer to induce corresponding virtual electrowetting locations on the surface of the first dielectric layer. Furthermore, means for manipulating the points of impingement of the electromagnetic radiation on the photoactive layer are provided and configured so as to vary the disposition of the virtual electrowetting locations thereby creating at least one electrowetting pathway along which the microdroplets may be caused to move.

In some embodiments, the microfluidic space is part of a microfluidic chip configured to manipulate microdroplets via optically mediated electrowetting (oEWOD).

In some embodiments, the microfluidic chip of the present invention comprises an oEWOD structure including first and second composite walls. The first composite wall may comprise: a first substrate; a first transparent conductor layer on the substrate, the first transparent conductor layer having a thickness in the range 70 to 250 nm; a photoactive layer activated by electromagnetic radiation in the wavelength range 400-850 nm on the conductor layer, the photoactive layer having a thickness in the range 300-1500 nm and a first dielectric layer on the photoactive layer, the first dielectric layer having a thickness in the range 30 to 160 nm. The second composite wall may comprise: a second substrate; a second conductor layer on the substrate, the second conductor layer having a thickness in the range 70 to 250 nm and optionally a second dielectric layer on the second conductor layer, the second dielectric layer having a thickness in the range 30 to 160 nm. The exposed surfaces of the first and second dielectric layers may be disposed 20-180 µm apart to define a microfluidic space adapted to contain microdroplets. An A/C source may further be included to provide a voltage across the first and second composite walls connecting the first and second conductor layers. The chip may further comprise first and second sources of electromagnetic radiation having an energy higher than the bandgap of the photoactive layer adapted to impinge on the photoactive layer to induce corresponding virtual electrowetting locations on the surface of the first dielectric layer. The chip may also include means for manipulating the points of impingement of the electromagnetic radiation on the photoactive layer so as to vary the disposition of the virtual electrowetting locations thereby creating at least one electrowetting pathway along which the microdroplets may be caused to move.

The first and second walls of these structures are transparent with the microfluidic space sandwiched in-between.

The first and second substrates are fabricated from any material which is mechanically strong enough to maintain the claimed geometry. For example: glass, metal or an engineering plastic. In some embodiments, the substrates may have a degree of flexibility. In yet another embodiment, the first and second substrates have a thickness in the range 100-1000 µm. In some embodiments, the first substrate is comprised of one of Silicon, fused silica, and glass. In some embodiments, the second substrate is comprised of one of fused silica and glass.

The first and second conductor layers are located on one surface of the first and second substrates and typically have a thickness in the range 70 to 250 nm, preferably 70 to 150 nm. At least one of these layers is made of a transparent conductive material such as Indium Tin Oxide (ITO), a very thin film of conductive metal such as silver or a conducting polymer such as PEDOT or the like. These layers may be formed as a continuous sheet or a series of discrete structures such as wires. Alternatively, the conductor layer may be a mesh of conductive material with the electromagnetic radiation being directed between the interstices of the mesh.

The photoactive layer may comprise a semiconductor material which can generate localised areas of charge in response to stimulation by the source of the second electromagnetic radiation. Examples include hydrogenated amorphous silicon layers having a thickness in the range 300 to 1500 nm. In some embodiments, the photoactive layer is activated by the use of visible light. The photoactive layer in the case of the first wall and optionally the conducting layer in the case of the second wall are coated with a dielectric layer which is typically in the thickness range from 30 to 160 nm. The dielectric properties of this layer preferably include a high dielectric strength of >10^7 V/m and a dielectric constant of >3. Preferably, it is as thin as possible consistent with avoiding dielectric breakdown. In some embodiments, the dielectric layer is selected from alumina, silica, hafnia or a thin non-conducting polymer film.

In another embodiment of these structures, at least the first dielectric layer, preferably both, are coated with an anti-fouling layer to assist in the establishing the desired microdroplet/carrier fluid/surface contact angle at the various virtual electrowetting electrode locations, and additionally to prevent the contents of the microdroplets adhering to the surface and being diminished as the microdroplet is moved through the chip. If the second wall does not comprise a second dielectric layer, then the second anti-fouling layer may be applied directly onto the second conductor layer.

For optimum performance, the anti-fouling layer should assist in establishing a microdroplet/carrier fluid/surface contact angle that should be in the range 50°-180° when measured as an air-liquid-surface three-point interface at 250° C. In some embodiments, these layer(s) have a thickness of less than 10 nm and are typically a monomolecular layer. In another, these layers are comprised of a polymer of an acrylate ester such as methyl methacrylate or a derivative thereof substituted with hydrophilic groups; e.g. alkoxysilyl. Either or both of the anti-fouling layers are hydrophobic to ensure optimum performance. In some embodiments an interstitial layer of silica of thickness less than 20 nm may be interposed between the anti-fouling coating and the dielectric layer in order to provide a chemically compatible bridge.

The first and second dielectric layers, and therefore the first and second walls, define a microfluidic space which is at least 10 µm, and preferably in the range of 20-180 µm, in width and in which the microdroplets are contained. Preferably, before they are contained, the microdroplets themselves have an intrinsic diameter which is more than 10% greater, suitably more than 20% greater, than the width of the microdroplet space. Thus, on entering the chip the microdroplets are caused to undergo compression leading to enhanced electrowetting performance through e.g. a better microdroplet merging capability. In some embodiments the first and second dielectric layers are coated with a hydrophobic coating such a fluorosilane.

In another embodiment, the microfluidic space includes one or more spacers for holding the first and second walls apart by a predetermined amount. Options for spacers include beads or pillars, ridges created from an intermediate resist layer which has been produced by photopatterning. Alternatively, deposited material such as silicon oxide or silicon nitride may be used to create the spacers. Alternatively layers of film, including flexible plastic films with or without an adhesive coating, can be used to form a spacer layer. Various spacer geometries can be used to form narrow channels, tapered channels or partially enclosed channels which are defined by lines of pillars. By careful design, it is possible to use these spacers to aid in the deformation of the microdroplets, subsequently perform microdroplet splitting and effect operations on the deformed microdroplets. Similarly these spacers can be used to physically separate zones of the chip to prevent cross-contamination between droplet populations, and to promote the flow of droplets in the correct direction when loading the chip under hydraulic pressure.

The first and second walls are biased using a source of A/C power attached to the conductor layers to provide a voltage potential difference therebetween; suitably in the range 10V to 50V. These oEWOD structures are typically employed in association with a source of second electromagnetic radiation having a wavelength in the range 400-850 nm, preferably 660 nm, and an energy that exceeds the bandgap of the photoactive layer. Suitably, the photoactive layer will be activated at the virtual electrowetting electrode locations where the incident intensity of the radiation employed is in the range 0.01 to 0.2 Wcm⁻².

Where the sources of electromagnetic radiation are pixelated they are suitably supplied either directly or indirectly using a reflective screen such as a digital micromirror device (DMD) illuminated by light from LEDs or other lamps. This enables highly complex patterns of virtual electrowetting electrode locations to be rapidly created and destroyed on the first dielectric layer thereby enabling the microdroplets to be precisely steered along essentially any virtual pathway using closely-controlled electrowetting forces. Such electrowetting pathways can be viewed as being constructed from a continuum of virtual electrowetting electrode locations on the first dielectric layer.

The points of impingement of the sources of electromagnetic radiation on the photoactive layer can be any convenient shape including the conventional circular or annular. In some embodiments, the morphologies of these points are determined by the morphologies of the corresponding pixilation and in another correspond wholly or partially to the morphologies of the microdroplets once they have entered the microfluidic space. In one embodiment, the points of impingement and hence the electrowetting electrode locations may be crescent-shaped and orientated in the intended direction of travel of the microdroplet. Suitably the electrowetting electrode locations themselves are smaller than the microdroplet surface adhering to the first wall and give a maximal field intensity gradient across the contact line formed between the droplet and the surface dielectric.

In some embodiments of the oEWOD structure, the second wall also includes a photoactive layer which enables virtual electrowetting electrode locations to also be induced on the second dielectric layer by means of the same or different source of electromagnetic radiation. The addition of a second dielectric layer enables transition of the wetting edge of a microdroplet from the upper to the lower surface of the structure, and the application of more electrowetting force to each microdroplet.

The first and the second dielectric layers may be composed of a single dielectric material or it may be a composite of two or more dielectric materials. The dielectric layers may be made from, but is not limited to, Al₂O₃ and SiO₂.

A structure may be provided between the first and second dielectric layers. The structure between the first and second dielectric layers can be made of, but is not limited to, epoxy, polymer, silicon or glass, or mixtures or composites thereof, with straight, angled, curved or micro-structured walls/faces. The structure between the first and second dielectric layers may be connected to the top and bottom composite walls to create a sealed microfluidic device and define the channels and regions within the device. The structure may occupy the gap between the two composite walls. Alternatively, or additionally, the conductor and dielectrics may be deposited on a shaped substrate which already has walls.

Some aspects of the methods and apparatus of the present invention are suitable to be applied to an optically-activated device other than an electrowetting device, such as a device configured to manipulate microparticles via dielectrophoresis or optical tweezers. In such a device cells or particles are manipulated and inspected using a functionally identical optical instrument to generate virtual optical dielectrophoresis gradients. Microparticles as defined herein may refer to particles such as biological cells, microbeads made of materials including polystyrene and latex, hydrogels, magnetic microbeads or colloids. Dielectrophoresis and optical tweezer mechanisms are well known in the art and could be readily implemented by the skilled person.

Similarly to the method described above for optical electrowetting, a first high-resolution optical assembly is used to perform fine manipulations and detailed inspection of the particles and/or cells through a combination of optically-mediated dielectrophoresis. A second coarse optical assembly is used to form an array of dielectrophoretic traps. The combination of these two assemblies gives the ability for the method to retain and transport a very large number of particles and/or cells using the coarse optical assembly, whilst performing fine manipulation and inspection operations using the fine optical assembly.

Thus, the disclosed microfluidic chip of the present invention advantageously allows for the manipulation of microdroplets across a wide range of sizes, and being digitally controlled, provides for dynamically re-programmable operational steps. The microfluidic substrates of the apparatus have no patterned electrodes, removing several complex low-yield fabrication steps and simplifying the electrical interconnections in comparison to conventional approaches. Device failures caused by dielectric breakdown between neighbouring electrodes are also eliminated thereby.

The resulting device structure thus permits more elaborate and integrated workflows compared to conventional approaches, such as independent control of the carrier phase and the droplets, as well as allowing for a greater density of droplets to be controlled across regions of the microfluidic chip surface. Up to one million droplets can be simultaneously manipulated.

The optical manipulation system can be combined with an integrated optical measurement and inspection system. This auxiliary system may allow the user to determine the content of the microdroplets including the number of cells, the number of microbeads, the brightness of any fluorescent signals and the morphology of the cells. By automated software recognition of the droplet contents it may be possible to enrich the droplets on the chip for a particular desired parameter using the oEWOD manipulation pattern to reject unwanted droplet to the outlet of the chip. As such it may be possible to form a population of droplets on the chip which contain exactly the correct number of carrier beads and cells to run the desired assay and growth steps.

The surface of the microbeads may be partially or fully coated. The microbeads can be coated with protein. In some embodiments, the microbeads have a surface functionalisation of a polypeptide configured to facilitate cell adhesion. In some embodiments, the microbead has a surface functionalisation of a polypeptide configured to facilitate cell adhesion.

In some embodiments, the peptide surface functionalisation comprises one or more of the following sequences; Gly-Arg-Gly-Asp-Ser (GRGDS), Arg-Gly-Asp (RGD) or Gly-Arg-Gly-Asp-Ser-Pro (GRGDSP). These sequences are short polypeptide sequences which are preferred as it minimise unwanted interferences on the surface of the microbead.

In some embodiments, the surface of the microbead may be coated with one or more of the following; a polypeptide, collagen, laminin, matrigel (RTM), synthetic hydrogel or polystyrene.

In some embodiments, the surface of the microbead may also be coated with a fouling reagent to form a fouling layer and promote culture growth and adhesion of target cells. The fouling agent may comprise Fetal Bovine Serum. In other embodiments, the fouling agent comprises a standard growth medium such as: F12 growth media, RPMI medium, DMEM, and Opti-MEM (RTM). In other embodiments, the fouling agent comprises one of: Green fluorescent protein, Bovine serum albumin, Fibronectin, Collagen, Laminin, Chitin, Matrigel (RTM), Hydrogel, and Elastin.

In other embodiments, the coating structure of the microbead may comprise at least one of Polylysine, (3-Aminopropyl)triethoxysilane (APTMS), Collagen, Laminin and Silicon dioxide.

In some embodiments the coating structure of the microbead may comprise one of Bovine Serum Albumin (BSA), Polylysine, Collagen, and Laminin, and forming the coating structure may comprise wetting the microbead with an aqueous solution comprising said compound such that the compound spontaneously, non-covalently adheres to the underlying surface.

In some embodiments the coating structure of the microbead may comprise a layer of BSA coupled to the surface via a chemical linker. In embodiments where the underlying surface exposes a layer of aluminium oxide, the chemical linker comprises 16-phosphonohexadecanoic acid or 3-Aminopropylphosphonic acid or any suitable ω-phosphonocarboxylic acids coupled to alkane chain linkers comprised of 3 to 16 (or more) methylene groups. In embodiments where the underlying surface exposes a layer of silicon dioxide, the chemical linker comprises (3-Aminopropyl) trimethoxysilane or a suitable ω-aminophosphonic acid coupled to an alkane chain comprised of 2-6 methylene groups. In some embodiments, coupling the protein to the aforementioned chemical linkers is done by simultaneously exposing both the BSA and the surface to N-(3-Dimethylaminopropyl)-N′-ethylcarbodiimide hydrochloride (EDC) such that covalent bonds form between the protein groups and the surface.

Alternatively, in some embodiments, a covalent bond is formed by first activating the surface using EDC in presence of N-Hydroxysulfosuccinimide sodium salt (sulfo-NHS), and then introducing the BSA in a subsequent step. Alternatively, such covalent bonds can be formed without the use of EDC, for example by using succinimidyl ester or succinic anhydride terminated linkers. In some embodiments the BSA is substituted for another appropriate protein such as collagen, laminin or fibronectin. In other embodiments the BSA is substituted with a mixture of appropriate proteins as detailed above.

In other embodiments, the coating structure of the microbead may comprise silicon dioxide, and forming the coating structure may comprise one of sputtering, atomic layer deposition or thermal evaporation thereof.

In some embodiments, the coating structure of the microbead may comprise a layer of antibodies to facilitate the adherence of antigens on the surface of target cells. The coating structure of the microbead is not limited to functionalisation with biologically active materials.

Preferably, the adherent cells can be in their native adherent state. In some embodiments, the adherent cell is in their native adherent state. Unless otherwise specified, the term “native state” as defined herein is referred to the physical and/or chemical properties of an adherent cell in its adherent state where it proliferates and adopts a stable phenotypic expression state. In the native adherent state, adherent cells are anchorage dependent and require attachment to a solid support for cell viability and cell growth. In some embodiments, the solid support can be a microbead. It is possible for adherent cells to be adapted for growth in suspension, however this requires the cells to be transitioned from an adherent state to a suspension state, and this process induces stresses to the cell, reduces the cell viability, and changes the expression profile of the cells. In some applications, adherent cells are required to grow in colonies in an adherent state after recovery. Therefore, it is highly advantageous to have a system where cells can be grown and recovered from a microfluidic system in their native adherent state. A system which cultures adherent cells in their native adherent state avoids having to temporarily transition cells back in to the suspension state for transportation out of the chip, and then return the cells to the adherent state for growth in microwell plates or flasks, and therefore avoids stresses to the cell.

In some embodiments, the method may further comprise the step of inspecting the contents of the first plurality and the second plurality of microdroplets to determine the number of beads and cells per droplet.

In some embodiments, the method may further comprise the step of inspecting at least a subset of the first plurality and the second plurality of microdroplets, prior to merging, to determine the contents of the microdroplets and the number of beads or cells per microdroplet.

In some embodiments, the method may further comprise the step of sorting operation configured to discard microdroplets except for those having a desired cell count. In some embodiments, the method may further comprise the step of a sorting operation to discard one or more microdroplets except for those having a desired cell count. The number of desired cells required to maintain clonality is one single cell.

In some embodiments, the method may further comprise the step of a sorting operation to discard microdroplets except for those having a desired bead count. In some embodiments, the method may further comprise the step of a sorting operation to discard one or more microdroplets except for those having a desired bead count. A desired bead count may be in a range of 1 to 10, or it may be more than 2, 4, 6 or 8. In some embodiments, the desired bead count may be less than 10, 8, 6, 4 or 2.

In some embodiments, the method may further comprise the step of identifying a selection of microdroplets having a bead count below a predetermined threshold and merging two or more of the selected droplets to increase the bead count. In some embodiments, the method may further comprise the step of identifying a selection of microdroplets having a bead count below a predetermined threshold and merging two or more of the selected microdroplets to increase the bead count.

The predetermined threshold value can vary from assay to assay. As an example, a microdroplet containing 1 to 10 microbeads may be selected to merge, or 3 to 10 microbeads may be selected to merge. In another example, a microdroplet containing 10 to 30 microbeads, 10 to 40, 10 to 50, 10 to 60, 10 to 80 microbeads may be selected to merge. Optionally, a microdroplet containing 1 to 100 microbeads may be selected to merge. In a further example, a microdroplet containing 100 to 200 microbeads may be selected to merge. In a further example, a microdroplet containing 100 to 300, 100 to 400, 100 to 500, 100 to 600, 100 to 700, 100 to 800 or 100 to 900 microbeads may be selected to merge. Optionally, a microdroplet containing above 1000 microbeads may be selected for merging operation. The merging operation may take place using oEWOD or EWOD.

In some embodiments, the method may further comprise the step of identifying a selection of microdroplets having a bead count at a pre-determined threshold level and splitting two or more of the selected microdroplets to decrease the bead count. The predetermined threshold value can vary from assay to assay. In this case and by way of example only, a microdroplet containing 1 to 10, 50, 100, 200, 300, 400, 500, 600, 700, 800, 900 or over 1000 microbeads may be selected for splitting. The splitting operation may take place using oEWOD or EWOD.

In some embodiments, the sorting and/or selection of microdroplets may be carried out under oEWOD control. Sorting droplets using oEWOD is advantageous because it can avoid the requirement for channels and/or valves to be used in sorting and selection operations. Sorting droplets under oEWOD control can therefore increase the efficiency of space used on the chip, and increase the speed of the microdroplet sorting operation.

In some embodiments, each of the first and/or second plurality of microdroplets may further comprise a coupling promoter.

One or more microdroplets may further comprise a coupling promoter such as 1-ethyl-3-(3-dimethylamino) propyl carbodiimide, hydrochloride (EDC). The coupling promoter is a crosslinking agent which activates carboxyl groups on the bead or protein coating and allows them to form covalent bonds with amide groups on the cell or another protein. A higher density of activated carboxyl groups gives a stronger bond.

In some embodiments, the method may further comprise the step of introducing a replacement carrier phase to the microfluidic space, the carrier phase having been equilibrated with cell growth media. Example of cell growth media are, but not limited to, RPMI 1640, EMEM, DMEM, Ham’s F12, Ham’s F10, F12-K, HAT Medium.

In some embodiments, the first fluid and/or second fluid may comprise cell growth media, and the method may further comprise the step of introducing a carrier phase to the microfluidic space, the carrier phase having been equilibrated with cell growth media, and wherein the carrier phase replaces any cell growth media depleted from the merged microdroplets.

A replacement carrier phase may comprise a fluid that is continuously exchanging with the surrounding oil. As the cells grow in the droplets the cells deplete the local environment of oxygen and/or carbon dioxide via the carrier phase. Key nutrients and supply of gases that promote cell growth such as oxygen and carbon dioxide gases can be dissolved in the carrier phase. In some embodiments, oxygen, carbon dioxide and other gases that are important for cell growth are continually replaced in the carrier phase to replenish the medium.

Equilibrating the carrier phase with cell growth media and/or oxygen and carbon dioxide can be advantageous because it enables the user to control the pH. Additionally, the carrier phase may have a small but sufficient capacity to dissolve water and cell media. By pre-treating the oil with cell media, the droplets can become more stable.

In some embodiments, a the cell growth medium that may be used include, but is not limited, to RPMI 1640, EMEM, DMEM, Ham’s F12, Ham’s F10, F12-K and/or HAT Medium.

In some embodiments, the carrier phase may additionally comprise a release agent.

In some embodiments, the carrier phase may comprise a release agent for releasing at least one adherent cell from at least one microbead.

The term ‘release agent,’ as used within the context of the present application, includes any substance which promotes the detachment of adherent cells from the solid support to which they have previously been adhered. The release agent promotes the release of adherent cells from their support by breaking the cell-support interactions, whilst causing minimal damage to the cell.

In some embodiments, the release agent may be one or more of the following: trypsin, EDTA, protease, citric acid or Accutase (RTM).

In some embodiments, the release agent may be one or more of the following: trypsin, EDTA, protease or citric acid.

Introduction of a release agent during the assay allows the cells to optionally be returned from the adherent state and back in to a suspension state. Advantageously, cells in a suspension state can then be subjected to further manipulations including division of the cell population between two daughter droplets formed by a droplet splitting operation for further sub-culturing. Furthermore cells in suspension state can be sampled in a daughter droplet as a sub-selection of cells and recovered off-chip for further analysis.

In some embodiments, the method may further comprise the step of incubating the merged droplets and monitoring cells adhering to the beads. In some embodiments, the method may further comprise the step of incubating the plurality of merged microdroplets and monitoring the cell adhering to the microbead in each of the merged microdroplets. The incubation may take place at 37° C., at atmospheric pressure with a composition of 5% CO₂, 21% O₂ and greater than 95% relative humidity.

Such monitoring includes brightfield microscopic inspection in order to determine the cell morphology and to count the number of cells. Similarly a fluorescence image or darkfield image can be taken in order to determine the phenotypic properties of cells via their chemical composition or the measurement of fluorescence reporters. Fluorescence reporters include endogenous reporter systems, in which cells express fluorescent proteins which can be measured by microscopic inspection. It also includes exogenous fluorescent reporters which may be specific to material on the cell surface or within the cell body; fluorescent images showing accumulation of exogenous reporters in the vicinity of the cell can similarly indicate a particular phenotypic state. The phenotypic state of a cell can depend heavily on whether or not it is in an adherent state. As such it is advantageous for profiling many important processes such as mammalian cell bioproduction if it is possible to monitor the phenotypic state of a cell whilst it is in an adherent conformation.

In some embodiments, the method may further comprise the step of performing an on-chip reporter assay on the merged microdroplets. For example, a report assay may include the use of attaching a fluorescent reporter onto the merged microdroplets for detection. This can be important for detecting adherent cells in merged droplets where the cells have been modified such that it can only be assayable in their adherent state i.e. some cells may only start secreting cytokines after they adhere to the microbead.

In some embodiments, the method may further comprise the step of dispensing the merged microdroplets into a receptacle. In some embodiments, the receptacle may be a well plate such as a tissue culture treated well plates. In some embodiments, the method may further comprise the step of dispensing the merged microdroplets into one or more tissue culture treated well plates. This step can be advantageous because allows a user to obtain the cells out of the droplets, onto the beads and then onto a tissue culture treated well plate without any extra processing steps involved.

In some embodiments, the method may further comprise the step of providing the beads and cells contained in the merged microdroplets to deposit onto a surface of the treated well plates such that the cells adhere to the surface and proliferate. Advantageously, in this embodiment cells can be recovered to treated well plates and spread from the bead to the plate surface without leaving the adherent state. In this embodiment a cluster of adherent cells (or a single adherent cell) on a bead is deposited in close proximity to a surface suitable for cell adhesion. In the case the bead is within a microdroplet, this deposition could be through printing, a spotting process or through dispensing the droplets through an orifice on to a surface. After the bead and cell(s) meet the surface, cells may spontaneously adhere on to the surface. Furthermore cells may proliferate and may adhere to the surface through the course of this proliferation. Advantageously, this embodiment eliminates the requirement to re-suspend cells, removing them from beads, before depositing them on to a culturing surface.

In some embodiments, the method may further comprise the step of depositing the plurality of merged microdroplets onto the surface of the treated well plate, wherein each merged microdroplets containing at least one adhered cell to at least one microbead.

The invention will now be further and more particularly described, by way of example only, and with reference to the accompanying drawings, in which:

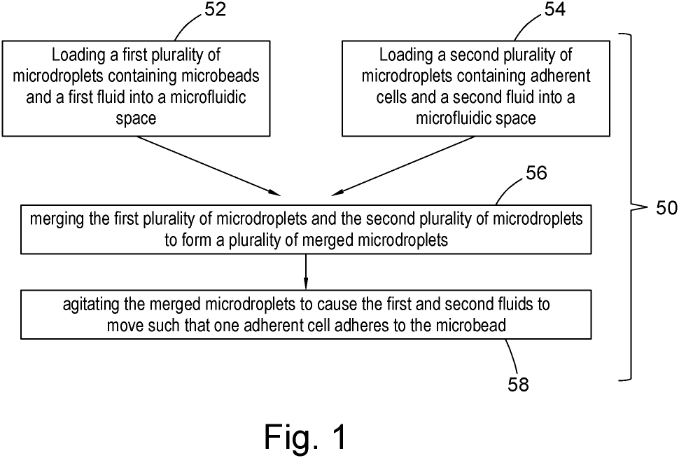

FIG. 1 provides a flowchart showing the method of the present invention;

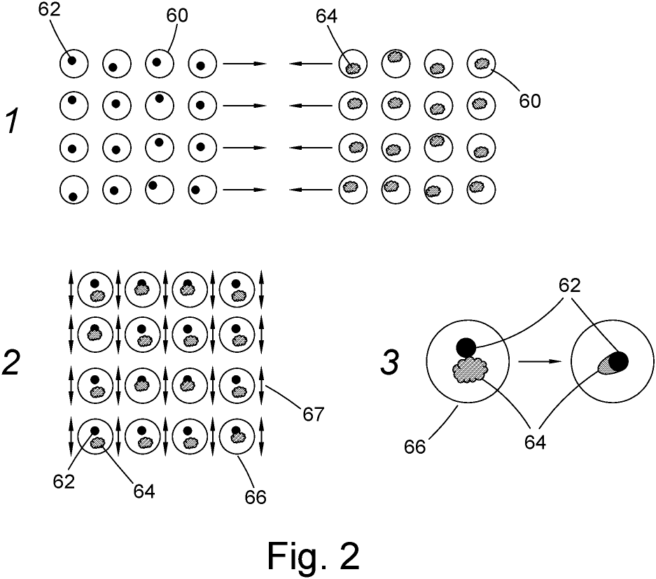

FIG. 2 shows a schematic of microdroplets containing microbeads and microdroplets containing adherent cells;

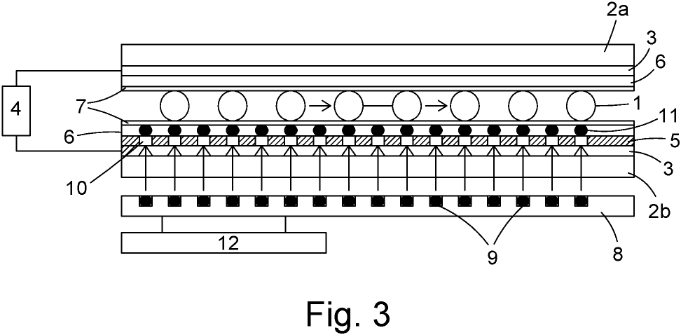

FIG. 3 shows an example configuration for carrying out the method of the present invention on a microfluidic chip;

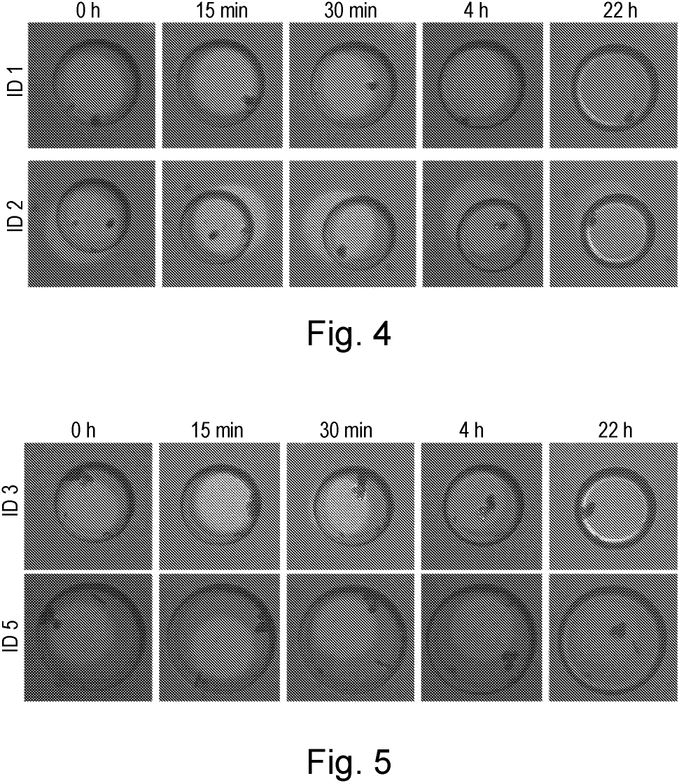

FIG. 4 showing a single cell adhered to a single microbead;

FIG. 5 showing multiple microbeads and multiple cells in a microdroplet;

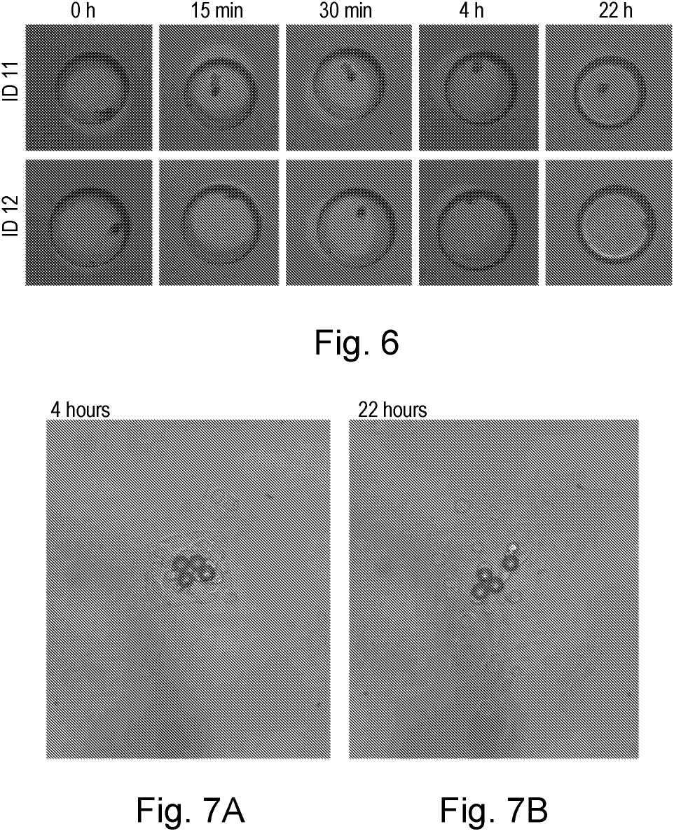

FIG. 6 showing a single microbead and multiple cells in a microdroplet;

FIGS. 7A and 7B illustrates cell viability after binding with microbeads at 4 and 22 hours, respectively; and

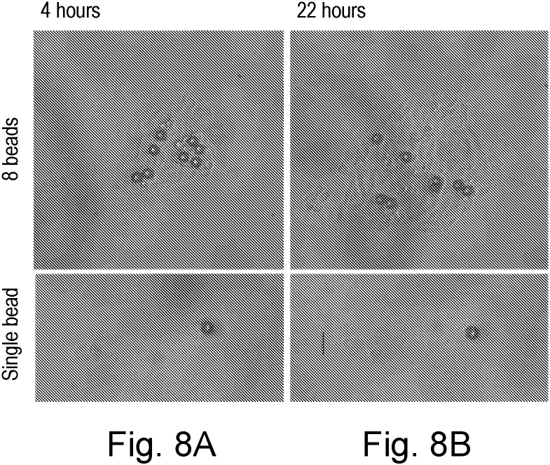

FIGS. 8A and 8B show cell proliferation on microbeads at 4 hours and 22 hours, respectively.

Referring to FIGS. 1 and 2, there is shown and illustrated a method of handling cells 50 such as adherent cells in a microdroplet assaying system. The method comprises conjugating adherent cells to microbeads. Referring to FIG. 1, a first plurality of microdroplets containing microbeads and a first fluid are loaded into a microfluidic space 52. The microfluidic space is part of a microfluidic chip configured to manipulate microdroplets via optically mediated electrowetting (oEWOD). A second plurality of microdroplets containing adherent cells and a second fluid are also be loaded into a microfluidic space 54. The second microdroplets can be loaded onto the same microfluidic chip as the first microdroplets, and can be positioned adjacent to the first microdroplets containing the adhered cells. The first and second plurality of microdroplets can be loaded into the microfluidic space via capillary action or it can be loaded into the microfluidic space via pressure driven flow. In some instances, a pump or a syringe may be used to load the first and/or second plurality of microdroplets into the microfluidic space.

In the next step, the first plurality of microdroplets merges with the second plurality of microdroplets 56. The second microdroplets may, for example, be caused to pair up with the first microdroplets in two paired microdroplet arrays. The second microdroplets are then caused to merge with the first microdroplets to form a plurality of merged microdroplets. Subsequently, each merged microdroplet contains the first and second fluids, at least one microbead and at least one adherent cell.

As shown FIG. 1, the method involves the step of agitating the merged microdroplets 58 via by stirring or by shaking or by any other force that is suitable to cause the first and second fluids to move such that at least one adherent cell moves towards at least one microbead and adheres itself to the microbead. Preferably, the merged microdroplets are stirred until the cells and beads come into contact and adhesion starts. The microdroplet of interest is then incubated and the process of cells adhering to the microbead is monitored.

The microbead may have a diameter of between 2 µm to 200 µm, but it may be more than 10, 20, 40, 80, 100, 120, 140, 160 or 180 µm. In some embodiments, the diameter of the microbead may be less than 200, 180, 160, 140, 120, 100, 80, 40, 20 or 10 µm. In some instances, two or more microbeads may come together to form bead clusters. The formation of bead clusters may occur by changing media conditions such as changing the pH and/or salt content of the media. Beads can cluster together to form a larger surface area such that it provides a bigger area for more adherent cells to adhere to.

Additionally, a surface of the microbeads can be coated and/or functionalised with a protein such as a short polypeptide. Examples of polypeptides sequence may include, but is not limited to, GRGD, RGD, GRGDS, or GRGDSP. The microbead can be partially or fully coated with a polypeptide. The polypeptide attached to the surface of the microbead can facilitate cell adhesion. Additionally or alternatively, the surface of the microbead can be coated with one or more of the following materials i.e. collagen, laminin and/or polystyrene.

The material of the microbead may be made from one or more of the following materials: Silica, polystyrene, latex, polyester, PMMA, magnetite and/or ferrite.

In one example, microbeads may be prepared with a surface functionalisation of a short peptide such as Gly-Arg-Gly-Asp-Ser (GRGDS). The short peptide i.e. Gly-Arg-Gly-Asp-Ser (GRGDS) can be aliquoted at 100 ug/mL in coupling buffer (Coupling buffer: 0.1 M MES, 0.5 M NaCl, pH 5.5). EDC can be immediately poured into the bead slurry. The beads are then vortex and incubate at room temperature for approximately 2 hours on a rotator. Occasionally the mix can be vortexed during incubation. The beads are then washed and resuspended in 1x PBS, 0.1% tween 20 and 0.02% NaN3, pH 7.4. The procedure as outlined above provides a microbead coated with a protein sequence of GRGDS. Microbeads may be coated with other protein sequences such as GRD.

Referring to FIG. 2, there is shown a method according to the present invention. As shown in FIG. 2, beads 62 in droplets 60 are merged with cells 64 in droplets 60 on an optofluidic device. After merging, the combined droplets 66 are agitated as indicated by the arrows 67, causing the beads 62 and the cells 64 to interact physically. The clusters of beads 62 and cells 64 are incubated inside the droplets and the cells enter their adherent state. The cluster can be inspected to see that the cell morphology has changed to be characteristic of the adherent state.

Preparation of an emulsion of the microbeads requires beads to first be suspended in a solution of cell media, before they are pumped under pressure through an emulsification apparatus. The volume bead density in the initial solution must match the required bead density in the resulting emulsion and the beads must be agitated to maintain a homogenous dispersion throughout the emulsification process. The emulsification apparatus comprises a microchannel plate chip with outlet orifices at one end, and an inlet for continuous fluid at the other end. The outlet end is immersed in to a vessel of carrier phase; typically this is an oil-based carrier phase, immiscible with the bead media. Beads pumped through the plate emerge surrounded by media at the outlet orifices where the media breaks off in to droplets. The resulting emulsion of media droplets containing beads surrounded by carrier phase can then be pumped in to an optofluidic chip for use in cell-based assays.

Preparation of an emulsion of cells requires recovering cells from an off-chip culture stock which is typically maintained in flat-bottomed cell culture flasks. Cells which are culturing in their adherent state must be resuspended using trypsin or another suitable release agent. The release agent must then be deactivated or removed in order not to inhibit subsequent return to adherent state; removal can be achieved by repeated washing steps in which cells are spun down to the bottom of a vessel in a centrifuge and the supernatant replaced with media. Deactivation can be achieved by the addition of excess protein substrate to the solution containing a protease-based release reagent. In the case that a particular occupancy of cells inside each droplet is required, the input must be diluted or concentrated such that the density of cells in the input matches the required droplet occupancy. Once the cells have been suspended and are at the required density and the release reagent has been either deactivated or removed, the cells must be pumped through an emulsification apparatus as described above for the microbeads.

The resulting emulsion is then pumped on to the optofluidic chip for droplet manipulation and the formation of adherent clusters of cells and beads.

At least one merged microdroplet containing at least one adherent cell adhered to at least one microbead may be selected for further assays. Some example assays have been performed on such cultured adherent cells, such as, for example, the introduction of a fluorescent reporter dye to the cultured adherent cells. Other example assays that could be performed on the cultured adherent cells may include: the introduction of a reporter bead, the introduction of a FRET reporter, the imaging of an endogenously expressed reporter, microscopic cell morphology measurements, lysis of the cultured cells, genetic detection assays such as PCR, isothermal amplification or fluorescence in-situ hybridisation, and DNA sequencing preparation. Alternatively the detached cells can simply be flowed off-chip for further analysis.

Referring to FIG. 3, there is shown an example configuration of a microfluidic chip comprising an oEWOD stack suitable for carrying out methods according to the present invention as disclosed.

Typically, microfluidic devices for manipulating droplets may cause the droplets, for example in the presence of an immiscible carrier fluid, to travel through a microfluidic space defined by two opposed walls of a cartridge or microfluidic tubing. Embedded within one or both walls are microelectrodes covered with a dielectric layer each of which is connected to an A/C biasing circuit capable of being switched on and off rapidly at intervals to modify the electric field characteristics of the layer. This gives rise to localised directional capillary forces in the vicinity of the microelectrodes which can be used to steer the droplet along one or more predetermined pathways. Such devices are known by the acronym EWOD (Electrowetting on Dielectric) devices. A variant of this approach, in which the electrowetting forces are optically-mediated optically-mediated, is known in the art as optoelectrowetting and hereinafter the corresponding acronym oEWOD.

Microfluidic devices employing oEWOD may include a microfluidic cavity defined by first and second walls and wherein the first wall is of composite design and comprised of substrate, photoconductive and insulating (dielectric) layers. Between the photoconductive and insulating layers, there may be disposed of an array of conductive cells which are electrically isolated from one another and coupled to the photoactive layer and whose functions are to generate corresponding electrowetting electrode locations on the insulating layer. At these locations, the surface tension properties of the droplets can be modified by means of an electrowetting field. These conductive cells may then be temporarily switched on by light impinging on the photoconductive layer. This approach has the advantage that switching is made much easier and quicker although its utility is to some extent still limited by the arrangement of the electrodes. Furthermore, there is a limitation as to the speed at which droplets can be moved and the extent to which the actual droplet pathway can be varied.

The example device as shown in FIG. 3 is suitable for the manipulation of aqueous microdroplets 1 having been emulsified into a fluorocarbon oil, having a viscosity of 1 centistokes or less at 25° C. and which in their unconfined state have a diameter of less than 100 µm e.g. in the range 20 to 80 µm. In some embodiments, the diameter may be more than 20, 30, 40, 50, 60, 80, 100, 120, 140, 160 or 180 µm. In some embodiments, the diameter may be less than 200, 180, 160, 140, 120, 100, 80, 60, 50, 30, 30 or 20 µm.

The oEWOD stack of the device comprises top 2a and bottom 2b glass plates each 500 µm thick coated with transparent layers of conductive Indium Tin Oxide (ITO) 3 having a thickness of 130 nm. Each of the layers of conductive Indium Tin Oxide (ITO) 3 is connected to an A/C source 4 with the ITO layer on bottom glass plate 2b being the ground. Bottom glass plate 2b is coated with a layer of amorphous silicon 5 which is 800 nm thick. Top glass plate 2a and the layer of amorphous silicon 5 are each coated with a 160 nm thick layer of high purity alumina or Hafnia 6 which are in turn coated with a monolayer of poly(3-(trimethoxysilyl)propyl methacrylate) 7 to render the surfaces of the layer of high purity alumina or Hafnia 6 hydrophobic.

Top glass plate 2a and the layer of amorphous silicon 5 are spaced 8 µm apart using spacers (not shown) so that the microdroplets undergo a degree of compression when introduced into the device cavity. An image of a reflective pixelated screen, illuminated by an LED light source 8 is disposed generally beneath bottom glass plate 2b and visible light (wavelength 660 or 830 nm) at a level of 0.01 Wcm2 is emitted from each diode 9 and caused to impinge on the layer of amorphous silicon 5 by propagation in the direction of the multiple upward arrows through bottom glass plate 2b and the layer of conductive Indium Tin Oxide (ITO) 3.

At the various points of impingement, photoexcited regions of charge 10 are created in the layer of amorphous silicon 5 which induce modified liquid-solid contact angles on the layer of high purity alumina or Hafnia 6 at corresponding electrowetting locations 11. These modified properties provide the capillary force necessary to propel the microdroplets 1 from one electrowetting location 11 to another. LED light source 8 is controlled by a microprocessor 12 which determines which of the diodes 9 in the array are illuminated at any given time by preprogrammed algorithms.

Further specific details of microfluidic chips suitable for carrying out the methods of the present invention may be found in the published patent WO 2018/234445, which is herein incorporated by reference.

The device of the present invention also provides for implementing environment controls suitable for the adherent cell conditions such as: controlled temperature, regions of different flow, controlling the carrier fluid to continuously feed cultured cells a supply of nutrients, and control of the local gas concentration in the carrier fluid surrounding the cultured cells.

For example, the adherent cell culture may be located in a region of low flow and surrounded by regions of faster flow that contain and supply nutrients and chemicals to the culture to encouraging growth.

Referring to FIG. 4, there is shown a single microbead and a single adherent cell inside a merged microdroplet. Each stage is taken at approximately 0 hours, 15 minutes, 30 minutes, 4 hours to 22 hours to show adherence of the adherent cell to the microbead. As time progresses, the single microbead and the single adherent cell come together into contact. The single adherent cell adheres to the microbead within the microdroplet as shown in FIG. 4. The arrow as indicated in FIG. 4 shows proliferation of adherent cells.

Referring to FIG. 5, there is shown a merged microdroplet containing multiple microbeads and multiple adherent cells. The merged microdroplet is monitored at each stage from 0 hours, 15 minutes, 30 minutes, 4 hours and 22 hours. FIG. 5 multiple adherent cells covering the microbeads and aggregate to one or more microbeads. In some instances, cell morphology such as structure, size and/or shape may change during the incubation period with the microbead. Changes in morphology are indicative of the phenotypic state of the cells and the growth process; it can be used as an indicator of cell health and viability.

Referring to FIG. 6, there is shown a merged microdroplet containing a single bead with multiple adherent cells. The merged microdroplet of interest is monitored at each stage from 0 hours, 15 minutes, 30 minutes, 4 hours and 22 hours. FIG. 6 shows that multiple adherent cells are able to adhere to a single microbead. In some instances, cell morphology such as structure, size and/or shape may change during the incubation period with the microbead.

FIGS. 7A and 7B provide images of a microdroplet at 4 hours and 22 hours respectively, showing cell viability tests after the cells have conjugated with the microbeads. The conditions provided enable the adherent cells to conjugate with the microbeads and the cells are left to culture on the microbeads for 4 hours. The microdroplet containing adherent cells and microbeads are then extracted and dispensed onto a tissue culture treated plate as shown in FIG. 7A. The cells are then left to culture on the microbeads for up to 22 hours. After 22 hours, the microdroplet containing adherent cells and microbeads are then extracted and dispensed onto a tissue culture treated plate as shown in FIG. 7B.

The results shown in FIG. 8A, which provides an image of a merged microdroplet at 4 hours and in FIG. 8B, which provides an image of the microdroplet at 22 hours, show that adherent cells on a single bead or on multiple beads are viable and able to proliferate during the incubation period.

Each droplet containing a plurality of cells and one or more microbeads may be manipulated according to the needs of particular sampling assays in any number of ways. Such manipulation may comprise altering the electrowetting conditions for the microdroplets such that the microdroplets de-wet or partially de-wet from the surface. The term “de-wet” as used herein refers to the change in contact angle between the droplet and the chip surface such that the droplet is pulled away from the surface.

Biological and/or chemical assays could be performed on the cultured cells that can include the introduction of a reporter bead, the introduction of a FRET reporter, the imaging of an endogenously expressed reporter, microscopic cell morphology measurements, lysis of the cultured cells, genetic detection assays such as PCR, isothermal amplification or fluorescence in-situ hybridisation, and DNA sequencing preparation. Alternatively the detached cells can simply be flowed off-chip for further analysis.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1-19. (canceled)

20. A method of handling an adherent cell in a microdroplet assaying system by conjugating an adherent cell to a microbead, the method comprising:
loading a first plurality of microdroplets into a microfluidic space, wherein each of the first microdroplet contains a microbead and a first fluid;
loading a second plurality of microdroplets into the microfluidic space, wherein each of the second microdroplet contains an adherent cell and a second fluid;
merging the first plurality of microdroplets and the second plurality of microdroplets to form a plurality of merged microdroplets, each merged microdroplets containing the first and second fluids, at least one microbead and at least one adherent cell; and
agitating each of the merged microdroplets to cause the first and second fluids in each of the merged microdroplets to move such that at least one adherent cell adheres to at least one microbead.

21. The method according to claim 20, further comprising the step of performing a selection, assaying, culturing or recovery process on the at least one adherent cell adhered to the at least one microbead.

22. The method according to claim 20, wherein the microfluidic space is part of a microfluidic chip configured to manipulate the first and second plurality of microdroplets via optically mediated electrowetting (oEWOD).

23. The method according to claim 20, wherein the microbead has a surface functionalisation of a polypeptide configured to facilitate cell adhesion.

24. The method according to claim 23, wherein the peptide surface functionalisation comprises one or more of the following sequences; Gly-Arg-Gly-Asp-Ser (GRGDS), Arg-Gly-Asp (RGD) or Gly-Arg-Gly-Asp-Ser-Pro (GRGDSP).

25. The method according to claim 20, wherein the adherent cell is in its native adherent state.

26. The method according to claim 20, wherein the method further comprises the step of inspecting at least a subset of the first plurality and the second plurality of microdroplets, prior to merging, to determine the contents of the microdroplets and the number of beads or cells per microdroplet.

27. The method according to claim 26, wherein the method further comprises the step of sorting operation configured to discard one or more microdroplets except for those having a desired cell count.

28. The method according to claim 26, wherein the method further comprises the step of sorting operation to discard one or more microdroplets except for those having a desired bead count.

29. The method according to claim 20, wherein the method further comprises the step of identifying a selection of microdroplets having a bead count below a predetermined threshold and merging two or more of the selected microdroplets to increase the bead count.

30. The method according to claim 20, wherein the method further comprise the step of identifying a selection of microdroplets having a bead count at a pre-determined threshold level and splitting two or more of the selected microdroplets to decrease the bead count.

31. The method according to claim 20, wherein each of the first and/or second plurality of microdroplets further comprise a coupling promoter.

32. The method according to claim 20, wherein the first fluid and/or second fluid comprises cell growth media and the method further comprises the step of introducing a carrier phase to the microfluidic space, the carrier phase having been equilibrated with cell growth media, and wherein the carrier phase is configured to replace cell growth media depleted from the merged microdroplets.

33. The method according to claim 32, wherein the carrier phase comprises a release agent for releasing at least one adherent cell from at least one microbead.

34. The method according to claim 33, wherein the release agent is one or more of the following; trypsin, EDTA, protease or citric acid.

35. The method according to claim 20, wherein the method further comprises the step of incubating the plurality of merged microdroplets and monitoring the cell adhering to the microbead in each of the merged microdroplets.

36. The method according to claim 20, wherein the method further comprises the step of performing an on-chip reporter assay on the merged microdroplets.

37. The method according to claim 20, wherein the method further comprises the step of dispensing the merged microdroplets into a receptacle.

38. The method according to claim 37, wherein the method further comprises the step of depositing the plurality of merged microdroplets onto the surface of the treated well plate, wherein each merged microdroplets containing at least one adhered cell to at least one microbead.


SEQUENCE LISTING (3 sequences)

SEQ ID NO: 1 - protein, 5 aa, Artificial Sequence
  GRGDS Sequence
GRGDS

SEQ ID NO: 2 - protein, 6 aa, Artificial Sequence
  GRGDSP Sequence
GRGDSP

SEQ ID NO: 3 - protein, 4 aa, Artificial Sequence
  GRGD Sequence
GRGD